(19) United States
(12) Patent Application Publication
(10) Pub. No.: US 2026/0354928 A1
(43) Pub. Date: Nov. 12, 2026

(54) Skin Markers and Sheets Thereof

(71) Applicant: Brady Worldwide, Inc., Milwaukee, WI (US)

(72) Inventors: Amanda R. Beekman, Hubertus, WI (US); Melissa C. Putzer, West Bend, WI (US); Jon Dable, Cudahy, WI (US)

(21) Appl. No.: 19/671,607

(22) Filed: May 8, 2026

Related U.S. Application Data
(60) Provisional application No. 63/802,157, filed on May 8, 2025.

(51) Int. Cl. A61B 90/00; A61B 6/04

(52) U.S. Cl. CPC A61B 90/39; A61B 6/0492; A61B 2090/3937

(57) ABSTRACT

The present disclosure provides a skin marker. The skin marker includes (A) a substrate; and (B) an adhesive layer comprising a pressure sensitive adhesive in contact with the substrate. In one embodiment, skin marker has (i) a water vapor transmission rate of at least 100 g/m²/day and (ii) an opacity of at least 65%. In another embodiment, the skin marker has (i) a breathability of at least 1.0 grams mass lost after 24 hours at 50 °C and (ii) an opacity of at least 65%. The present disclosure also provides a sheet containing a plurality of skin markers in contact with a release liner.

### BACKGROUND

The present disclosure is directed to skin markers and sheets thereof.

The present skin markers are useful as markers that may be placed on the skin of an individual undergoing radiation treatment to visually indicate the precise location of the area to be treated. The location of the treatment areas can take significant time to identify-time of the patient, doctors, and other medical professionals. Consequently, it is inefficient to locate treatment areas multiple times. Yet, radiation therapy can go on for weeks and the precise location of the area to be treated must be identifiable during the entire treatment period.

A longstanding problem in marking radiation therapy treatment areas is that doctors and other medical professionals require the marker to be visible and identifiable for a span of several weeks (e.g., a six-week treatment). Attempts have been made to use permanent tattoos on patients to mark treatment areas, but patients can be unwilling to receive a permanent tattoo for various reasons, such as religious objections or for cosmetic reasons (for example, if the location is on the patient's face). Additionally, if the treatment area changes over time, the presence of permanent tattoos marking old treatment areas may cause confusion or errors in treatments.

Other attempts have been made to use temporary tattoos on patients to mark treatment areas. However, temporary tattoos can be difficult to keep in place over the span of several weeks. Consequently, medical professionals have begun covering the temporary tattoos using adhesive composites (e.g., Tegaderm), tape, wound spray, stickers, or the like, to protect the temporary tattoo. These can be uncomfortable for a patient to wear on their skin long-term because, for example, the adhesive of the temporary tattoo covers picks up lint and the temporary tattoo covers are not breathable or flexible enough for long-term wear on a person's skin. Repeated removal and application of a temporary tattoo cover for radiation therapy treatments can irritate the patient's skin. Further, these temporary tattoo covers are very noticeable on a person's skin, which may cause a patient to be self-conscious or uncomfortable. While some attempts have been made to use transparent or substantially transparent temporary tattoo covers, the temporary tattoos are visible through the cover, which can, themselves, be noticeable.

The art recognizes a need for a skin marker for identifying radiation therapy treatment areas that avoids the need for a permanent tattoo on a patient's skin, while is also visible for the duration of a radiation therapy treatment. The art further recognizes the need for a skin marker for identifying radiation therapy treatment areas that is comfortable for a patient to wear for the duration of a radiation therapy treatment.

### SUMMARY

The present disclosure provides a skin marker. The skin marker includes (A) a substrate; and (B) an adhesive layer comprising a pressure sensitive adhesive in contact with the substrate. The skin marker has (i) a water vapor transmission rate of at least 100 g/m²/day, or at least 400 g/m²/day, or at least 500 g/m²/day, or at least 750 g/m²/day, or at least 850 g/m²/day and (ii) an opacity of at least 65%.

In an embodiment, the skin marker includes (A) a substrate; and (B) an adhesive layer comprising a pressure sensitive adhesive in contact with the substrate. The skin marker has (i) a water vapor transmission rate of at least 100 g/m²/day, or at least 400 g/m²/day, or at least 500 g/m²/day, or at least 750 g/m²/day, or at least 850 g/m²/day and (ii) an opacity of at least 65%.

In an embodiment, the skin marker includes (A) a substrate; and (B) an adhesive layer comprising a pressure sensitive adhesive in contact with the substrate. The skin marker has (i) a breathability of at least 1.0 grams mass lost after 24 hours at 50 °C and (ii) an opacity of at least 65%.

The present disclosure also provides a sheet including (A) a plurality of skin markers, each skin marker including (1) a substrate, and (2) an adhesive layer comprising a pressure sensitive adhesive in contact with the substrate, each skin marker having (i) a water vapor transmission rate of at least 100 g/m²/day, or at least 400 g/m²/day, or at least 500 g/m²/day, or at least 750 g/m²/day, or at least 850 g/m²/day and (ii) an opacity of at least 65%; and (B) a release liner in contact with the adhesive layer of each of the plurality of the skin markers.

In an embodiment, the sheet includes (A) a plurality of skin markers, each skin marker including (1) a substrate, and (2) an adhesive layer comprising a pressure sensitive adhesive in contact with the substrate, each skin marker having (i) a water vapor transmission rate of at least 100 g/m²/day, or at least 400 g/m²/day, or at least 500 g/m²/day, or at least 750 g/m²/day, or at least 850 g/m²/day and (ii) an opacity of at least 65%; and (B) a release liner in contact with the adhesive layer of each of the plurality of the skin markers.

In an embodiment, the sheet includes (A) a plurality of skin markers, each skin marker including (1) a substrate, and (2) an adhesive layer comprising a pressure sensitive adhesive in contact with the substrate, each skin marker having (i) a breathability of at least 1.0 grams mass lost after 24 hours at 50 °C and (ii) an opacity of at least 65%; and (B) a release liner in contact with the adhesive layer of each of the plurality of the skin markers.

### BRIEF DESCRIPTION OF THE DRAWINGS

FIG. 1A is a schematic of a skin marker in accordance with an embodiment of the present disclosure.

FIG. 1B is a schematic of a skin marker in accordance with another embodiment of the present disclosure.

FIG. 2 is a schematic of a skin marker in accordance with another embodiment of the present disclosure.

FIG. 3 is a schematic of a skin marker in accordance with another embodiment of the present disclosure.

FIG. 4 is a perspective view of a skin marker in accordance with an embodiment of the present disclosure.

FIG. 5 is a top plan view of an X-shaped skin marker in accordance with an embodiment of the present disclosure.

FIG. 6 is a top plan view of an irregular-shaped skin marker in accordance with an embodiment of the present disclosure.

FIG. 7 is a top plan view of a symbol-shaped skin marker in accordance with an embodiment of the present disclosure.

FIG. 8 is a top plan view of a rectangular-shaped skin marker with an X-shaped cutout in accordance with an embodiment of the present disclosure.

FIG. 9 is a top plan view of a rectangular-shaped skin marker with three X-shaped cutouts in accordance with an embodiment of the present disclosure.

FIG. 10 is a top plan view of a rectangular-shaped skin marker with an X-shaped cutout and a graphical image in accordance with an embodiment of the present disclosure.

FIG. 11 is a schematic of an X-shaped skin marker and a rectangular-shaped skin marker with an X-shaped cutout positioned on a person's skin in accordance with an embodiment of the present disclosure.

FIG. 12 is a top plan view of a squircle-shaped skin marker in accordance with an embodiment of this present disclosure.

FIG. 13 is a top plan view of a squircle-shaped skin marker with a circle-shaped cutout in accordance with an embodiment of the present disclosure.

FIG. 14 is a top plan view of a sheet of skin markers in accordance with an embodiment of the present disclosure.

FIG. 15 is a top plan view of a sheet of skin markers in accordance with another embodiment of the present disclosure.

FIG. 16A is a top plan view of a sheet of skin markers in accordance with an embodiment of the present disclosure.

FIG. 16B is a top plan view of a sheet of skin markers in accordance with another embodiment of the present disclosure.

FIG. 17 is a top plan view of a sheet of skin markers in accordance with another embodiment of the present disclosure

### DEFINITIONS AND TEST METHODS

For purposes of United States patent practice, the contents of any referenced patent, patent application or publication are incorporated by reference in their entirety (or its equivalent US version is so incorporated by reference) especially with respect to the disclosure of definitions (to the extent not inconsistent with any definitions specifically provided in this disclosure) and general knowledge in the art.

The numerical ranges disclosed herein include all values from, and including, the lower and upper value. For ranged containing explicit values (e.g., 1 or 2; or 3 to 5; or 6; or 7), any subrange between any two explicit values is included (e.g., 1 to 2; 2 to 6; 5 to 7; 3 to 7; 5 to 6; etc.).

Unless stated to the contrary, implicit from the context, or customary in the art, all parts and percents are based on weight and all test methods are current as of the filing date of this disclosure.

Any reference to the Periodic Table of Elements is that as published by CRC Press, Inc., 1990-1991. Reference to a group of elements in this table is by the new notation for numbering groups.

The terms “comprising,” “including,” “having,” and their derivatives, are not intended to exclude the presence of any additional component, step, or procedure, whether or not the same is specifically disclosed. In order to avoid any doubt, all articles claimed through use of the term “comprising” may include any additional component, feature, or element, unless stated to the contrary. In contrast, the term, “consisting essentially of” excludes from the scope of any succeeding recitation any other component, step, or procedure, excepting those that are not essential to operability. The term “consisting of” excludes any component, step, or procedure not specifically delineated or listed. The term “or,” unless stated otherwise, refers to the listed members individually as well as in any combination. Use of the singular includes use of the plural and vice versa. As used herein, “a,” “an,” “the,” “at least one,” and “one or more” are used interchangeably.

The term “contact” includes direct contact and indirect contact.

“Direct contact” refers to a layer configuration whereby a first layer is located immediately adjacent to a second layer and no intervening layers or no intervening structures are present between the first layer and the second layer.

“Graphic image” and like terms mean text or pictorial representations formed of ink or other dye or pigments substances. Graphic images include, but are not limited to, words, numbers, bar codes, pictures, designs (geometric or otherwise), and solid colors (typically applied by flood coating).

“Indirect contact” refers to a layer configuration whereby a first layer is located adjacent to a second layer and at least one intervening layer or intervening structure is present between the first layer and the second layer.

“Ink” and like terms mean a coatable or printable formulation that can and usually does contain a dye and/or pigment. Nonlimiting examples of suitable ink is inkjet ink, pen ink, wax ink, or marker ink. In some embodiments, the ink is a ultraviolet (“UV”) ink or a water-based ink.

A “layer” is a single thickness or coating of a compound, polymer, or composition spread out or covering a surface.

A “marker” is a shape of material, typically stored on a sheet or roll, having a top layer and an adhesive layer.

“Nonwoven” refers to a web or fabric having a structure of individual fibers or threads which are randomly interlaid, but not in an identifiable manner as is the case of a knitted fabric. The following descriptions provide nonlimiting procedures for producing nonwoven fabric. Fibers produced by melt spinning processes that include staple fiber spinning (including short spinning, long spinning), Spunbond, melt blown, or multiple combinations thereof, can be formed into a web, which is thereafter formed into a nonwoven fabric using binding technologies, such as carded thermal bonding, wetlaid, airlaid, airthrough bonding, calendar thermal bonding, hydro entanglement, needlepunching, adhesive bonding, or any combinations thereof.

The term “parallel,” as used herein, refers to structures or axes extending in the same direction along the length of the structures or direction of the axes, wherein a spatial distance between the structures or axes does not change along the length of the structures or direction of the axes.

The term “perpendicular,” as used herein, refers to structures or axes extending in directions that intersect at a 90°, or substantially a 90° angle along the length of the structures or direction of the axes.

A “person,” “wearer,” “patient,” or an “individual” each refer to either a human person or an animal. A nonlimiting example is a human person undergoing radiation therapy.

“Pigment” refers to a visible light absorbing material or compound that is present in a non-molecularly dispersed (particulate) form.

“Planar surface”, “facial surface”, “top surface”, “bottom surface” and the like are used in distinction to “edge surface.” If rectangular in shape or configuration, a marker will have two opposing planar surfaces joined by four edge surfaces (two opposing pairs of edge surfaces, each pair intersecting the other pair at right angles). If circular in configuration, then the marker will have two opposing planar surfaces joined by one continuous edge surface. While the markers of this invention may be limited to a defined range of thickness, the planar dimensions of the marker can be of any length and/or width and shape and as such, so can the planar and edge surfaces, e.g., polygonal or circular, flat or wavy, etc. Typically, the marker is of conventional planar dimensions, e.g., from 0.25 to 15 inches, or from 0.50 to 10 inches, or from 0.50 to 5 inches, or from 0.50 to 2 inches, in width with a length of from 0.25 to 15 inches, or from 0.50 to 10 inches, or from 0.50 to 5 inches, or from 0.50 to 2 inches. In another embodiment, from 0.25 to 15 inches, or from 0.50 to 10 inches, or from 0.50 to 5 inches, or from 0.50 to 2 inches, in width with a length of 0.25, 0.50, 1, 3, 5, 10, 20, 30, 40, 50, 100, 150, 200, or more inches wound and stored in roll form on a cylindrical tube, or in sheet form. Top surface and bottom surface are relative terms and in the context of this invention, the bottom surface of a layer is the surface closest to the wearer's skin when the marker is applied to the wearer, e.g., a patient undergoing radiation therapy, and the top surface of a layer is the surface distal to the wearer's skin when the marker is applied to the wearer's skin.

A “polygon” is a closed-plane figure bounded by at least three sides. The polygon can be a regular polygon, or an irregular polygon having three, four, five, six, seven, eight, nine, ten, or more sides. Nonlimiting examples of suitable polygonal shapes include triangle, square, rectangle, diamond, trapezoid, parallelogram, hexagon, and octagon.

A “polyamide” is a polymer with repeating units linked by amide bonds. Nonlimiting examples of polyamides include aliphatic polyamides (e.g., nylon), polythalamides, and aromatic polyamides (e.g., poly-paraphenylene terephthalamide (i.e., Kevlar™)).

A “polymer” is a macromolecular compound prepared by polymerizing monomers of the same or different type. “Polymer” includes homopolymers, copolymers, terpolymers, interpolymers, and so on. An “interpolymer” is a polymer prepared by the polymerization of at least two types of monomers or comonomers. It includes, but is not limited to, copolymers (which usually refers to polymers prepared from two different types of monomers or comonomers, terpolymers (which usually refers to polymers prepared from three different types of monomers or comonomers), tetrapolymers (which usually refers to polymers prepared from four different types of monomers or comonomers), and the like.

A “pressure sensitive adhesive” or “PSA” is a type of adhesive composition that forms a bond with a surface, such as a wearer's skin or a layer surface, upon the application of pressure.

A “printable” surface is a surface, or marker, that is configured to run through a printing machine in its normal operation, and receive and retain ink on or in the top facial surface. To constitute a printable skin marker, the skin marker must have a structure that is capable of being run through a printing machine (e.g., having an appropriate thickness). Further, the skin marker's top facial surface (for example, the top surface of the substrate) should be smooth, or substantially smooth, so as to avoid uneven printing or voids in printing. Moreover, in some embodiments, the skin marker should have a substantially uniform thickness from each edge surface to the opposing edge surface.

“Spunbond” and like terms, as used herein, mean a fabric or like material that is made by depositing extruded, spun filaments onto a collecting belt in a uniform, random manner followed by bonding of the fibers.

“Translucent” as used in the context of this invention includes transparent.

Thickness is measured in accordance with ASTM D1000-17.

Breathability is measured in accordance with ASTM E96/96M-22 (63.5 mm diameter test area).

Water vapor transmission rate (“WVTR”) is measured in accordance with ASTM E96, using the Cup Test Method, and specifically, the Desiccant Cup Method.

### DETAILED DESCRIPTION

The present disclosure provides a skin marker. The skin marker includes (A) a substrate; and (B) an adhesive layer comprising a pressure sensitive adhesive in contact with the substrate. The skin marker has (i) a water vapor transmission rate (“WVTR”) of at least 100 g/m²/day, or at least 400 g/m²/day, or at least 500 g/m²/day, or at least 750 g/m²/day, or at least 850 g/m²/day and (ii) an opacity of at least 65%.

In an embodiment, the skin marker includes (A) a substrate; and (B) an adhesive layer comprising a pressure sensitive adhesive in contact with the substrate. The skin marker has (i) a WVTR of at least 100 g/m²/day and (ii) an opacity of at least 65%.

In an embodiment, the skin marker includes (A) a substrate; and (B) an adhesive layer comprising a pressure sensitive adhesive in contact with the substrate. The skin marker has (i) a breathability of at least 1.0 grams mass lost after 24 hours at 50 °C and (ii) an opacity of at least 65%.

In an embodiment, the skin marker includes (A) a substrate; and (B) an adhesive layer comprising a pressure sensitive adhesive in contact with the substrate. The skin marker has (i) a breathability of at least 2.0 grams mass lost after 24 hours at 50 °C and (ii) an opacity of at least 65%.

The present disclosure also provides a sheet including (A) a plurality of skin markers, each skin marker including (1) a substrate, and (2) an adhesive layer comprising a pressure sensitive adhesive in contact with the substrate, each skin marker having (i) a WVTR of at least 100 g/m²/day, or at least 400 g/m²/day, or at least 500 g/m²/day, or at least 750 g/m²/day, or at least 850 g/m²/day and (ii) an opacity of at least 65%; and (B) a release liner in contact with the adhesive layer of each of the plurality of the skin markers.

In an embodiment, the sheet includes (A) a plurality of skin markers, each skin marker including (1) a substrate, and (2) an adhesive layer comprising a pressure sensitive adhesive in contact with the substrate, each skin marker having (i) a WVTR of at least 100 g/m²/day and (ii) an opacity of at least 65%; and (B) a release liner in contact with the adhesive layer of each of the plurality of the skin markers.

In an embodiment, the sheet includes (A) a plurality of skin markers, each skin marker including (1) a substrate, and (2) an adhesive layer comprising a pressure sensitive adhesive in contact with the substrate, each skin marker having (i) a breathability of at least 1.0 grams mass lost after 24 hours at 50 °C, or at least 2.0 grams mass lost after 24 hours at 50 °C, or at least 8.0 grams mass lost after 24 hours at 50 °C and (ii) an opacity of at least 65%; and (B) a release liner in contact with the adhesive layer of each of the plurality of the skin markers.

### A. Substrate

The skin marker includes a substrate.

In an embodiment, the substrate is a fabric substrate or a film.

In an embodiment, the substrate is a fabric substrate. The fabric substrate may comprise any natural and/or synthetic materials. Synthetic materials include, for example, various synthetics based on polyolefins (e.g., polyethylene, polypropylene, etc.), nylon, polyester, polyurethane (e.g., a spandex material), and blends or combinations thereof. Natural materials include, for example, cotton, silk, wool (e.g., merino wool or alpaca wool), or blends thereof. In a particular embodiment, the fabric substrate is a nonwoven, or further, a spunbound. In an alternative embodiment, the fabric substrate layer is a woven material.

In an alternative embodiment, the substrate layer is an expanded polytetrafluoroethylene (ePTFE) sheet, such as Gore-Tex fabric, available from W. L. Gore & Associates, Inc.

In an embodiment, the substrate is a film. A nonlimiting example of a film is a polymeric film.

In an embodiment, the substrate is selected from silk, cotton, wool, hemp, yarn, rayon, a spunbound fabric, a polymer film, a nonwoven, or a combination thereof.

In an embodiment, the substrate is a nonwoven.

In an embodiment, the substrate is a nonwoven with fiber formed from polypropylene, polyester, polyimide (e.g., nylon), polyethylene, polylactic acid, or a combination thereof.

In an embodiment, the substrate is a nonwoven with fiber formed from polyester, nylon, polylactic acid, or a combination thereof. In a further embodiment, the fiber is formed from a blend of polyester and nylon. In another embodiment, the fiber is formed from polylactic acid (“PLA”).

In an embodiment, the substrate is a nonwoven with fiber formed from polyethylene. A nonlimiting example of a suitable nonwoven with fiber formed from polyethylene is Tyvek, available from DuPont.

In an embodiment, the substrate has a WVTR of at least 2,000 g/m²/day. In another embodiment, the substrate has a WVTR of at least 100 g/m²/day, or at least 500 g/m²/day, or at least 750 g/m²/day, or at least 1,000 g/m²/day, or at least 2,500 g/m²/day, or at least 3,000 g/m²/day, or at least 3,500 g/m²/day, or at least 4,000 g/m²/day, or at least 4,500 g/m²/day, or at least 5,000 g/m²/day. In another embodiment, the substrate has a WVTR of from 100 g/m²/day to 40,000 g/m²/day, or from 1,000 g/m²/day to 40,000 g/m²/day, or from 2,000 g/m²/day to 40,000 g/m²/day, or from 2,000 g/m²/day to 20,000 g/m²/day, or from 2,000 g/m²/day to 10,000 g/m²/day. In another embodiment, the substrate has a WVTR of from 100 g/m²/day, or 750 g/m²/day, or 1,000 g/m²/day, or 1,500 g/m²/day, or 2,000 g/m²/day, or 2,500 g/m²/day, or 3,000 g/m²/day, or 3,500 g/m²/day, or 4,000 g/m²/day, or 4,500 g/m²/day, or 5,000 g/m²/day to 10,000 g/m²/day, or 20,000 g/m²/day, or 30,000 g/m²/day, or 40,000 g/m²/day.

In an embodiment, the substrate has a breathability of at least 10 grams (g) mass lost after 24 hours at 50 °C. In a further embodiment, the substrate has a breathability of at least 10 g, or at least 15 g, or at least 20 g, or at least 25 g, or at least 30 g, or at least 35 g, or at least 40 g mass lost after 24 hours at 50 °C. In another embodiment, the substrate has a breathability of from 10 g to 100 g, or from 20 g to 100 g, or from 30 g to 100 g, or from 40 g to 100 g mass lost after 24 hours at 50 °C. In another embodiment, the substrate has a breathability of from 10 g, or 15 g, or 20 g, or 25 g, or 30 g, or 35 g, or 40 g to 50 g, or 100 g mass lost after 24 hours at 50 °C.

In an embodiment, the substrate has an opacity of at least 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%. In a further embodiment, the substrate has an opacity of from 10% to 100%, or from 20% to 100%, or from 30% to 100%, or from 40% to 100%, or from 50% to 100%, or from 60% to 100%, or from 70% to 100%, or from 80% to 100%, or from 90% to 100%. In another embodiment, the substrate has an opacity of from 10%, or 15%, or 20%, or 25%, or 30%, or 35%, or 40%, or 45%, or 50%, or 55%, or 60%, or 65%, or 70%, or 75%, or 80%, or 85%, or 90% to 95%, or 99%, or 100%.

In an embodiment, the substrate is not translucent. In other words, the substrate is opaque.

In an alternate embodiment, the substrate has an opacity of less than 50%, or less than 45%, or less than 40%, or less than 35%, or less than 30%, or less than 25%, or less than 20%, or less than 15%, or less than 10%, or less than 5%. In an embodiment, the substrate is translucent. In other words, the substrate is not opaque.

It is understood that while the substrate may have an opacity of less than 50%, the skin marker shall have an opacity of at least 65%.

In an embodiment, the substrate has a thickness of from 1 mil (25.4 μm) to 30 mil (762 μm). In a further embodiment, the substrate has a thickness of from 1 mil, or 2 mil, or 3 mil to 5 mil, or 8 mil, or 10 mil, or 12 mil, or 13 mil, or 14 mil, or 15 mil, or 16 mil, or 17 mil, or 18 mil, or 19 mil, or 20 mil, or 25 mil, or 30 mil, or 35 mil, or 40 mil, or 45 mil, or 50 mil. In another embodiment, the substrate has a thickness of from 1 mil to 50 mil, or from 1 mil to 40 mil, or from 1 mil to 30 mil, or from 1 mil to 25 mil, or from 1 mil to 20 mil, or from 1 mil to 15 mil, or from 3 mil to 15 mil.

Not wishing to be bound by any particular theory, it is believed that a substrate that is a nonwoven, or further a spunbound, which have a random arrangement of fibers, can provide a high WVTR and high breathability, while also providing sufficient opacity to prevent an individual from seeing through the skin marker. Additionally, it is believed that the random arrangement of fibers in the nonwoven, and further in a spunbound, disorient a viewer's eye, which makes it difficult to see unsightly ink or temporary tattoos positioned on an individual's skin, under the skin marker.

In some embodiments, the substrate is perforated.

In some embodiments, the substrate is continuous from one side edge to the opposing side edge. In other words, the substrate is not perforated, notched, or otherwise discontinuous.

In an embodiment, the substrate is chemically or physically treated to enhance the substrate's breathability. A nonlimiting example of a physical treatment is stretching the substrate. A nonlimiting example of a suitable chemical treatment is to cravenette the substrate, or to treat the substrate with oils or fluoropolymers. Alternatively, the substrate may include an optional additive to enhance the substrate's breathability, such as CaCO₃ or TiO₂.

In an embodiment, the substrate is a top layer of the skin marker. The top layer is the layer distal to a wearer's skin when the skin marker is applied to the wearer's skin.

In an embodiment, the substrate has one, some, or all of the following properties:
(a) the substrate is selected from silk, cotton, wool, hemp, yarn, rayon, a spunbound fabric, a polymer film, a nonwoven, or a combination thereof; and/or
(b) the substrate is a nonwoven; and/or
(c) the substrate is a nonwoven with fiber formed from polypropylene, polyester, polyimide (e.g., nylon), polyethylene, polylactic acid, or a combination thereof; and/or
(d) the substrate is a nonwoven with fiber formed from polyester, nylon, polylactic acid, or a combination thereof; and/or
(e) the substrate is a nonwoven with fiber formed from polyethylene;
(f) the substrate has a WVTR of at least 100 g/m²/day, or at least 750 g/m²/day, or at least 1,000 g/m²/day, or at least 2,500 g/m²/day, or at least 3,000 g/m²/day, or at least 3,500 g/m²/day, or at least 4,000 g/m²/day, or at least 4,500 g/m²/day, or at least 5,000 g/m²/day; and/or
(g) the substrate has a WVTR of from 100 g/m²/day to 40,000 g/m²/day, or from 1,000 g/m²/day to 40,000 g/m²/day, or from 2,000 g/m²/day to 40,000 g/m²/day, or from 2,000 g/m²/day to 20,000 g/m²/day, or from 2,000 g/m²/day to 10,000 g/m²/day; and/or
(h) the substrate has a breathability of at least 10 g, or at least 15 g, or at least 20 g, or at least 25 g, or at least 30 g, or at least 35 g, or at least 40 g mass lost after 24 hours at 50 °C; and/or
(i) the substrate has a breathability of from 10 g to 100 g, or from 20 g to 100 g, or from 30 g to 100 g, or from 40 g to 100 g mass lost after 24 hours at 50 °C; and/or
(j) the substrate has an opacity of at least 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%; and/or
(k) the substrate has an opacity of from 10% to 100%, or from 20% to 100%, or from 30% to 100%, or from 40% to 100%, or from 50% to 100%, or from 60% to 100%, or from 70% to 100%, or from 80% to 100%, or from 90% to 100%; and/or
(l) the substrate has a thickness of from 1 mil to 50 mil, or from 1 mil to 40 mil, or from 1 mil to 30 mil, or from 1 mil to 25 mil, or from 1 mil to 20 mil, or from 1 mil to 15 mil, or from 3 mil to 15 mil.

The substrate has a top facial surface and an opposing bottom facial surface. The top facial surface and the opposing bottom facial surface extend parallel, or substantially parallel, to each other.

In some embodiments, the top facial surface of the substrate contains a graphic image.

In an embodiment, the top facial surface of the substrate is printable, such as with an inkjet printer, a thermal printer, or a waxjet printer.

In an embodiment, the top facial surface of the substrate is coated with a topcoat, such as the topcoats the described in U.S. Publication No. 2011/0027513; U.S. Publication No. 2011/0268897; U.S. Pat. Nos. 8,936,841; 8,920,895; or U.S. Publication No. 2024/0010021, the entire contents of which are herein incorporated by reference. In some embodiments, the topcoat enables the top facial surface of the skin marker to be printable. In other embodiments, the topcoat may enable the skin marker to be more durable. In an embodiment, the skin marker includes a topcoat layer in contact, and further in direct contact, with a top surface of the substrate.

In an embodiment, the substrate is a nonwoven, or further a spunbound, and the fibers or threads include a dye or an ink. In other words, the extruded polymer fibers or threads are formed from a polymeric composition containing a dye or ink.

In an embodiment, the substrate includes a fiber or thread, or a plurality of fibers or threads, that are of a different color than the majority (the majority being greater than 50 wt %, based on the total weight of fibers or threads in the substrate) of the fibers or threads contained in the substrate. The different colored fibers or threads may form a graphical image on or in the substrate. For example, the different colored fibers or threads may form a crosshairs symbol visible from the top surface of the substrate.

The substrate may comprise two or more embodiments disclosed herein.

### B. Adhesive Layer

The skin marker includes an adhesive layer including a pressure sensitive adhesive (PSA) in contact with the substrate.

The adhesive layer has a top facial surface and an opposing bottom facial surface.

In an embodiment, the substrate directly contacts the adhesive layer. In other words, the bottom facial surface of the substrate directly contacts the top facial surface of the adhesive layer. In another embodiment, the substrate indirectly contacts the adhesive layer.

In an embodiment, the adhesive layer is configured to directly contact a person/wearer's skin when the skin maker is placed on the skin. In other words, the bottom facial surface of the adhesive layer is configured to directly contact a person/wearer's skin when the skin marker is placed on the person/wearer.

In an embodiment, the adhesive layer directly contacts the optional release liner, which is described in detail below. In other words, the bottom facial surface of the adhesive layer directly contacts a top facial surface of the release liner.

Nonlimiting examples of suitable pressure sensitive adhesives include acrylic-based PSAs, acrylic-hybrid PSAs, rubber-based PSAs, silicone-based PSAs, and combinations thereof. The PSA may be thermoplastic, solvent-based, or water-based.

In an embodiment, the PSA is an acrylic-based PSA. Acrylic-based PSAs are formed from an acrylic-based polymer. A nonlimiting example of a suitable acrylic-based PSA is AROSET PS 5333, available from Ashland Inc.

In an embodiment, the PSA is an acrylic-hybrid PSA. Nonlimiting examples of suitable acrylic hybrids include acrylic-rubber hybrid and acrylic-silicone hybrid PSAs. In an embodiment, the acrylic-hybrid PSA is an acrylic-rubber PSA.

In an embodiment, the PSA is a rubber-based PSA. Nonlimiting examples of suitable rubber-based PSAs include compositions containing (i) one or more polymers such as styrene-butadiene (SBR), styrenic block copolymer (such as polystyrene-polyisoprene-polystyrene (SIS) or polystyrene-polybutadiene-polystyrene (SBS)), and butyl rubber; and (ii) an optional tackifying resin such as polyisobutylene.

In an embodiment, the PSA is selected from an acrylic-based PSA, an acrylic-hybrid PSA, a rubber-based PSA, and combinations thereof.

In an embodiment, the PSA is an acrylic-based PSA.

In an embodiment, the PSA is a medical-grade PSA. A nonlimiting example of a medical-grade PSA is AROSET PS 5333, available from Ashland Inc.

In an embodiment, the PSA includes one or more optional additives. Nonlimiting examples of suitable additives are calcium carbonate and a pigment. A nonlimiting example of a pigment is titanium dioxide.

In an embodiment, the adhesive layer contains an additive selected from calcium carbonate, titanium dioxide, and combinations thereof.

In an embodiment, the adhesive layer contains up to 20 wt % additive, based on a total weight of the adhesive layer. In another embodiment, the adhesive layer contains from greater than 0 wt % to 20 wt %, or from 0.1 wt % to 20 wt %, or from 0.5 wt % to 15 wt %, or from 0.5 wt % to 10 wt %, or from 0.5 wt % to 5 wt % additive, based on a total weight of the adhesive layer.

In an embodiment, the adhesive layer has a thickness of from 1 mil, or 2 mil, or 3 mil, or 4 mil, or 5 mil, or 6 mil, or 10 mil to 12 mil, or 14 mil, or 15 mil, or 20 mil. In another embodiment, the adhesive layer has a thickness of from 1 mil to 20 mil, or from 1 mil to 15 mil, or from 1 mil to 12 mil, or from 2 mil to 12 mil, or from 5 mil to 12 mil, or from 6 mil to 12 mil.

In an embodiment, the adhesive layer has a substantially uniform thickness from each edge surface to the opposing edge surface. In an adhesive layer having substantially uniform thickness from each edge surface to the opposing edge surface, the top facial surface and the opposing bottom facial surface extend parallel, or substantially parallel, to each other.

In an embodiment, the adhesive layer has one, some, or all of the following properties:
(a) the PSA is a medical-grade PSA; and/or
(b) the PSA is selected from acrylic-based PSAs, acrylic-hybrid PSAs, rubber-based PSAs, silicone-based PSAs, and combinations thereof, and/or
(c) the PSA is an acrylic-based PSA; and/or
(d) the adhesive layer contains an additive selected from calcium carbonate, titanium dioxide, and combinations thereof, and/or
(e) the adhesive layer contains from greater than 0 wt % to 20 wt %, or from 0.1 wt % to 20 wt %, or from 0.5 wt % to 15 wt %, or from 0.5 wt % to 10 wt %, or from 0.5 wt % to 5 wt % additive, based on a total weight of the adhesive layer; and/or
(f) the adhesive layer has a thickness of from 1 mil to 20 mil, or from 1 mil to 15 mil, or from 1 mil to 12 mil, or from 2 mil to 12 mil, or from 5 mil to 12 mil, or from 6 mil to 12 mil; and/or
(g) the adhesive layer has a substantially uniform thickness from each edge surface to the opposing edge surface.

The skin marker may include one or more adhesive layers. The composition and properties of each adhesive layer may be the same or different. For example, the skin marker may have a first adhesive layer and a second adhesive layer. In a first embodiment, each of the first and second adhesive layers may be formed from an identical PSA composition and have identical thicknesses. In a second embodiment, the first adhesive layer may be formed from a different PSA composition than the second adhesive layer, and each of the first and second adhesive layers may have identical thicknesses. In a third embodiment, the first adhesive layer may be formed from a different PSA composition than the second adhesive layer, and the first and second adhesive layers may have different thicknesses relative to each other.

In an embodiment, the skin marker has a single, or one and only one, adhesive layer. The adhesive layer directly contacts the substrate.

In an embodiment, the skin marker has at least two adhesive layers (which may be the same or different in composition and thickness). At least one of the adhesive layers is configured to be the bottom layer of the skin marker. The bottom layer is the layer proximate to a person/wearer's skin when the skin marker is applied to the person/wearer's skin.

The adhesive layer may comprise two or more embodiments disclosed herein.

### C. Optional Release Liner

In an embodiment, the skin marker includes an optional release liner. The optional release liner directly contacts an adhesive layer.

Nonlimiting examples of release liners include glassine paper, laminated paper, polyester film, and polypropylene film, each of which may or may not have been subjected to a coating of silicone.

In one embodiment the skin marker is void of a release liner. In this embodiment the release liner may or may not be replaced with a release coating, e.g., a silicone-based or wax-based material.

A nonlimiting example of a suitable release liner is a fluoropolymer coated polyester release liner, such as 3M's product number 9956.

The release liner is removed from the skin marker before it is applied to a person/wearer's skin.

The release liner has a top facial surface and an opposing bottom facial surface.

In an embodiment, the release liner has a thickness of from 0.5 mil, or 1 mil, or 2 mil to 3 mil, or 4 mil, or 5 mil, or 6 mil, or 10 mil, or 12 mil, or 14 mil, or 15 mil. In another embodiment, the release liner has a thickness of from 0.5 mil to 15 mil, or from 1 mil to 10 mil, or from 1 mil to 5 mil, or from 2 mil to 4 mil, or from 3 mil to 4 mil, or from 2 mil to 3 mil.

The release liner may be a single continuous sheet, or may be a plurality of sheets of release liner that overlap with each other, or contact each other. In another embodiment, the release liner may be scored or perforated to facilitate the removal of the release liner from the adhesive layer. In a further embodiment, the release liner may have a fold line or a crease line to facilitate the removal of the release liner from the adhesive layer.

The optional release liner may comprise two or more embodiments disclosed herein.

### D. Skin Marker

The present disclosure provides a skin marker.

In an embodiment, the skin marker has (i) a WVTR of at least 100 g/m²/day and (ii) an opacity of at least 65%.

In an embodiment, the skin marker has (i) a WVTR of at least 400 g/m²/day and (ii) an opacity of at least 65%.

In an embodiment, the skin marker has (i) a WVTR of at least 500 g/m²/day and (ii) an opacity of at least 65%.

In an embodiment, the skin marker has (i) a WVTR of at least 750 g/m²/day and (ii) an opacity of at least 65%.

In an embodiment, the skin marker has (i) a WVTR of at least 850 g/m²/day and (ii) an opacity of at least 65%.

In an embodiment, the skin marker has (i) a breathability of at least 1.0 grams mass lost after 24 hours at 50 °C and (ii) an opacity of at least 65%.

In an embodiment, the skin marker has (i) a breathability of at least 2.0 grams mass lost after 24 hours at 50 °C and (ii) an opacity of at least 65%.

In an embodiment, the skin marker has (i) a breathability of at least 8.0 grams mass lost after 24 hours at 50 °C and (ii) an opacity of at least 65%.

In an embodiment, the skin marker has a WVTR of at least 100 g/m²/day, or at least 400 g/m²/day, or at least 500 g/m²/day, or at least 750 g/m²/day, or at least 850 g/m²/day. In another embodiment, the skin marker has a WVTR of at least 100 g/m²/day, or at least 400 g/m²/day, or at least 500 g/m²/day, or at least 750 g/m²/day, or at least 800 g/m²/day, or at least 900 g/m²/day, or at least 1000 g/m²/day, or at least 1200 g/m²/day, or at least 1500 g/m²/day, or at least 1800 g/m²/day. In a further embodiment, the skin marker has a WVTR of from 100 g/m²/day to 40,000 g/m²/day, or from 450 g/m²/day to 40,000 g/m²/day, or from 500 g/m²/day to 40,000 g/m²/day, or from 750 g/m²/day to 40,000 g/m²/day, or from 800 g/m²/day to 40,000 g/m²/day, or from 500 g/m²/day to 30,000 g/m²/day, or from 800 g/m²/day to 30,000 g/m²/day, or from 750 g/m²/day to 30,000 g/m²/day, or from 800 g/m²/day to 30,000 g/m²/day, or from 900 g/m²/day to 30,000 g/m²/day, or from 900 g/m²/day to 20 g/m²/day, or from 1000 g/m²/day to 40,000 g/m²/day, or from 1000 g/m²/day to 30,000 g/m²/day, or from 1500 g/m²/day to 30,000 g/m²/day, or from 1800 g/m²/day to 30,000 g/m²/day, or from 800 g/m²/day to 10,000 g/m²/day, or from 850 g/m²/day to 10,000 g/m²/day, or from 800 g/m²/day to 10,000 g/m²/day, or from 450 g/m²/day to 5,000 g/m²/day, or from 450 g/m²/day to 2,000 g/m²/day, or from 800 g/m²/day to 5,000 g/m²/day, or from 850 g/m²/day to 2,000 g/m²/day. In another embodiment, the skin marker has a WVTR of from 100 g/m²/day, or 450 g/m²/day, or 500 g/m²/day, or 700 g/m²/day, or 750 g/m²/day, or 800 g/m²/day, or 850 g/m²/day, or 900 g/m²/day, or 950 g/m²/day, or 1000 g/m²/day, or 1100 g/m²/day, or 1500 g/m²/day, or 1800 g/m²/day, or 2000 g/m²/day, or 2500 g/m²/day to 5,000 g/m²/day, or 10,000 g/m²/day, or 15,000 g/m²/day, or 20,000 g/m²/day, or 25,000 g/m²/day, or 30,000 g/m²/day, or 35,000 g/m²/day, or 40,000 g/m²/day.

In an embodiment, the skin marker has a breathability of at least 1.0 grams (g) mass lost after 24 hours at 50 °C. In some embodiments, the skin marker has a breathability of at least 1.0 g, or at least 1.05 g, or at least 2.0 g, or at least 3.0 g, or at least 5.0 g, or at least 8.0 g, or at least 9.0 g, or at least 10 g, or at least 11 g mass lost after 24 hours at 50 °C. In a further embodiment, the skin marker has a breathability of from 1.0 g to 50 g, or from 1.0 g to 40 g, or from 1.0 g to 30 g, or from 1.0 g to 20 g, or from 2 g to 20 g, or from 5 g to 20 g, or from 5 g to 30 g, or from 5 g to 40 g, or from 5 g to 50 g, or from 10 g to 50 g, or from 10 g to 40 g, or from 10 g to 30 g, or from 10 g to 20 g mass lost after 24 hours at 50 °C. In another embodiment, the skin marker has a breathability of from 1.0 g, or 1.01 g, or 1.05 g, or 2.0 g, or 3.0 g, or 4.0 g, or 5.0 g, or 8.0 g, or 10 g, or 11 g to 15 g, or 20 g, or 25 g, or 30 g, or 35 g, or 40 g, or 45 g, or 50 g mass lost after 24 hours at 50 °C.

Not wishing to be bound by any particular theory, it is believed that a skin marker that does not have at least one of (i) a breathability of at least 1.0 grams (g) mass lost after 24 hours at 50 °C, or at least 2.0 g mass lost after 24 hours at 50 °C or (ii) a WVTR of at least 100 g/m²/day, or at least 400 g/m²/day, or at least 500 g/m²/day, or at least 750 g/m²/day, or at least 850 g/m²/day, when worn for a time period of from several days to several weeks, will leave the underlying skin wet, distorted, or pruney, and may result in skin having a higher transepidermal water loss (“TEWL”), which is the amount of water that evaporates from the body surface. Increased TEWL has been associated with skin changes caused by radiation dermatitis. Skin with higher TEWL may also be more vulnerable to irritation or experience a loss of barrier function. Further, wet, sweaty, distorted, or pruney skin may cause premature failure of the adhesive holding a skin marker in place. Moreover, higher breathability and higher WVTR results in less sweat accumulating around and under the skin marker. Sweat accumulation can distort or wash away any ink present on a wearer's skin, rendering any ink markings on the person's skin unreadable.

The skin marker has an opacity of at least 65%. In some embodiments, the skin marker has an opacity of at least 67%, or at least 70%, or at least 75%, or at least 80%, or at least 85%, or at least 90%, or at least 91%, or at least 95%, or at least 97%. In further embodiments, the skin marker has an opacity of from 65% to 100%, or from 67% to 100%, or from 70% to 100%, or from 75% to 100%, or from 80% to 100%, or from 85% to 100%, or from 90% to 100%, or from 95% to 100%. In another embodiment, the skin marker has an opacity of from 65%, or 67%, or 70%, or 75%, or 80%, or 85%, or 90%, or 95%, or 97% to 99%, or 100%. It is understood that the opacity of the skin marker is measured without the optional release liner. In other words, if a release liner is present, the release liner is removed before opacity is measured.

In an embodiment, the skin marker has substantially uniform opacity, or uniform opacity, from each edge surface to the opposing edge surface. It is understood that a skin marker having substantially uniform opacity, or uniform opacity, from each edge surface to the opposing edge surface excludes any cutaway (or cutout) that may be present within the skin marker's outer perimeter.

In an embodiment, the skin marker has an opacity of at least 65%, or at least 67%, or at least 70%, or at least 75%, or at least 80%, or at least 85%, or at least 90%, or at least 91%, or at least 95%, or at least 97% across an entire surface area of the skin marker, or from each edge surface to the opposing edge surface. In other words, the skin marker is void of, or excludes, areas or portions that have an opacity of less than 65%.

In an embodiment, the skin marker has a total thickness of from 2 mil to 100 mil, or from 2 mil to 50 mil, or from 2 mil to 40 mil, or from 2 mil to 30 mil, or from 2 mil to 25 mil, or from 2 mil to 20 mil, or from 5 mil to 20 mil, or from 5 mil to 25 mil, or from 9 mil to 20 mil, or from 9 mil to 25 mil. In another embodiment, the skin marker has a total thickness of from 2 mil, or 3 mil, or 4 mil, or 5 mil, or 6 mil, or 7 mil, or 8 mil, or 9 mil to 10 mil, or 12 mil, or 15 mil, or 20 mil, or 25 mil, or 30 mil, or 35 mil, or 40 mil, or 45 mil, or 50 mil, or 100 mil. In an embodiment, the skin marker has a total thickness of from 2 mil to 20 mil. It is understood that the total thickness of the skin marker excludes the thickness of the optional release liner.

In an embodiment, the skin marker has a surface area of at least 0.25 in², or at least 0.5 in². In another embodiment, the skin marker has a surface area of from 0.25 in² to 100 in², or from 0.25 in² to 50 in², or from 0.25 in² to 25 in², or from 0.25 in² to 10 in², or from 0.25 in² to 5 in², or from 0.25 in² to 1 in². In a further embodiment, the skin marker has a surface area of from 0.25 in², or 0.50 in² to 1 in², or 2 in², or 5 in², or 10 in², or 25 in², or 50 in², or 100 in².

In an embodiment, the skin marker has an opacity of at least 65%, or at least 70%, or at least 75%, or at least 80%, or at least 85%, or at least 90%, or at least 91%, or at least 95%, or at least 97%; and the skin marker has at least one of (A) a WVTR of at least 100 g/m²/day, or at least 400 g/m²/day, or at least 500 g/m²/day, or at least 750 g/m²/day, or at least 800 g/m²/day, or at least 900 g/m²/day, or at least 1000 g/m²/day, or at least 1200 g/m²/day, or at least 1500 g/m²/day, or at least 1800 g/m²/day; or (B) a breathability of at least 1.0 g, or at least 1.05 g, or at least 2.0 g, or at least 3.0 g, or at least 5.0 g, or at least 8.0 g, or at least 9.0 g, or at least 10 g, or at least 11 g mass lost after 24 hours at 50 °C. In some embodiments, the skin marker has a total thickness of from 2 mil to 50 mil, or from 2 mil to 40 mil, or from 2 mil to 30 mil, or from 2 mil to 25 mil, or from 2 mil to 20 mil, or from 5 mil to 20 mil, or from 5 mil to 25 mil, or from 9 mil to 20 mil, or from 9 mil to 25 mil. In some embodiments, the skin marker has substantially uniform opacity from each edge surface to the opposing edge surface.

In an embodiment, the skin marker has an opacity of at least 65%, or at least 70%, or at least 75%, or at least 80%, or at least 85%, or at least 90%, or at least 91%, or at least 95%, or at least 97%; and the skin marker has at least one of (A) a WVTR of at least 100 g/m²/day, or at least 400 g/m²/day, or at least 500 g/m²/day, or at least 750 g/m²/day, or at least 800 g/m²/day, or at least 900 g/m²/day, or at least 1000 g/m²/day, or at least 1200 g/m²/day, or at least 1500 g/m²/day, or at least 1800 g/m²/day; or (B) a breathability of at least 2.0 g, or at least 3.0 g, or at least 5.0 g, or at least 8.0 g, or at least 9.0 g, or at least 10 g, or at least 11 g mass lost after 24 hours at 50 °C. In some embodiments, the skin marker has a total thickness of from 2 mil to 20 mil, or from 5 mil to 20 mil, or from 9 mil to 20 mil, or from 2 mil to 17 mil. In some embodiments, the skin marker has substantially uniform opacity from each edge surface to the opposing edge surface.

In an embodiment, the skin marker includes (A) the substrate and (B) the adhesive layer containing the PSA in contact with the substrate. In a further embodiment, the skin marker has the following Structure I:

Substrate/Adhesive Layer (Structure I).

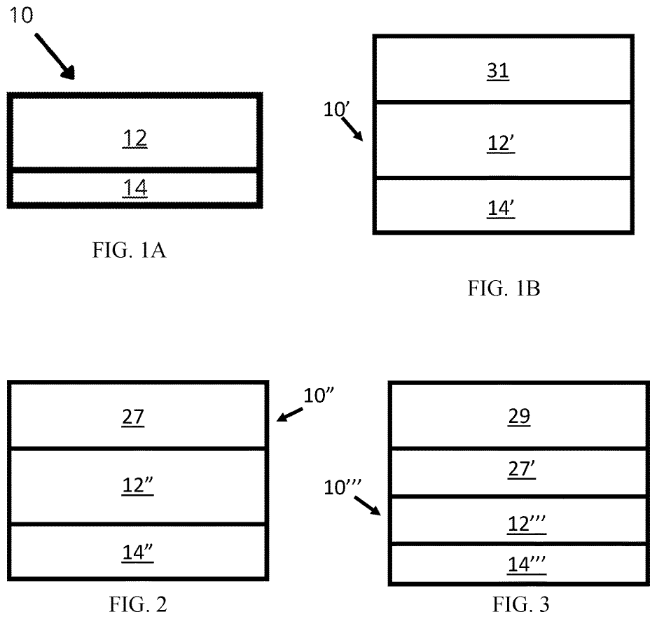

FIG. 1A is a schematic of a skin marker having the Structure I. The skin marker 10 has a substrate 12 and an adhesive layer 14. The substrate 12 directly contacts the adhesive layer 14.

In an embodiment, the skin marker includes (A) the substrate; (B) the adhesive layer including a PSA in contact with the substrate; and (C) an optional release liner in direct contact with the adhesive layer. In a further embodiment, the skin marker has the following Structure II:

Substrate/Adhesive Layer/Optional Release Liner (Structure II).

In an embodiment, the skin marker includes (A) the substrate; (B) the adhesive layer including a PSA in contact with the substrate; and (C) an optional topcoat in direct contact with the substrate. In a further embodiment, the skin marker has the following Structure III:

Optional Topcoat/Substrate/Adhesive Layer/Optional Release Liner (Structure III).

In an embodiment, the skin marker includes (A) the substrate; (B) the adhesive layer including a PSA in contact with the substrate; (C) an optional release liner in direct contact with the adhesive layer; and (D) an optional topcoat in direct contact with the substrate. In a further embodiment, the skin marker has the following Structure IV:

Optional Topcoat/Substrate/Adhesive Layer/Optional Release Liner (Structure IV).

FIG. 1B is a schematic of a skin marker having the Structure IV, without the optional release liner. The skin marker 10′ has a substrate 12′ and an adhesive layer 14′. The substrate 12′ directly contacts the adhesive layer 14′, and the topcoat 31 directly contacts the substrate 12′.

In some embodiments, the skin marker is coated with an ink to form an ink layer. The ink layer may be in contact with the substrate, or may be in contact with the optional topcoat. The ink layer may be continuous or discontinuous. A continuous ink layer is present at a substantially uniform thickness from each edge surface to the opposing edge surface of the skin marker. A discontinuous ink layer has gaps, or voids. A nonlimiting example of a discontinuous ink layer is an ink layer in the shape of a graphic image. A nonlimiting example of a suitable ink is a water-based black ink from Siegwerk Druckfarben AG & Co. KGaA.

In some embodiments, the optional ink layer has a skin-toned color, such that the skin marker appears skin-colored. A skin-colored ink marker advantageously detracts attention from the skin marker while it is being worn by an individual, making the individual less self-conscious and more comfortable. A nonlimiting example of a skin-toned color ink is a flesh tone UV-ink available from Sun Chemical.

In some embodiments, the skin marker may have a plurality, or more than one, or two optional ink layers. For example, the skin marker may be coated with a first ink layer that has a skin-toned color, the first ink layer in contact with the substrate. A second ink layer that is in contact with the first ink layer may be in another color, for example, black, and may be in the form of a graphical image, for example. In some embodiments, the first ink layer is continuous and the second ink layer is discontinuous.

It is understood that some or all of the ink may be absorbed by the substrate, or the ink may form a discrete layer having a top surface and an opposing bottom surface, the bottom surface contacting the substrate. Regardless of whether the ink is absorbed by the substrate (partially or completely), the ink deposited onto the coating layer is herein referred to as an “ink layer.”

The ink layer may directly or indirectly contact the substrate. In an embodiment, the ink layer, or at least one ink layer, directly contacts the substrate.

In some embodiments, the substrate has a white color. In a further embodiment, an optional ink layer having a skin-toned color contacts the substrate. The optional ink layer may be continuous or discontinuous. In one embodiment, the optional ink layer is discontinuous. For example, the optional ink layer may form an outline of a crosshairs symbol, such that the crosshairs symbol white (from the top surface of the substrate) is visible to a wearer or medical professional, and area outside the crosshairs symbol is skin-toned.

In an embodiment, the skin marker includes (A) the substrate; (B) the adhesive layer containing the PSA in contact with the substrate; and (C) an optional ink layer in contact with the substrate. In a further embodiment, the skin marker has the following Structure V:

Optional Ink Layer/Substrate/Adhesive Layer (Structure V).

FIG. 2 is a schematic of a skin marker having the Structure V. The skin marker 10″ has a substrate 12″, an adhesive layer 14″, and an ink layer 27. The substrate 12″ directly contacts the adhesive layer 14″, and the ink layer 27 directly contacts the substrate 12″.

In some embodiments, the skin marker contains at least two, or a plurality of ink layers. The ink layers may be identical or different. In some embodiments, the skin marker includes two ink layers, the first ink layer being a continuous layer in contact with the substrate, and the second ink layer being a graphic image in contact with the first ink layer. FIG. 3 is a schematic of a skin marker having two ink layers. The skin marker 10′″ has a substrate 12′″, an adhesive layer 14′″, a first ink layer 27″, and a second ink layer 29. The substrate 12′″ directly contacts the adhesive layer 14′″, the first ink layer 27″ directly contacts the substrate 12′″, and the second ink layer 29 directly contacts the first ink layer 27″.

It is understood that while FIGS. 2 and 3 depict the ink layers 27, 27′, 29 as distinct layers it is understood that the ink layers 27, 27′, 29 may or may not be absorbed by the substrate (partially or completely).

In an embodiment, the skin marker includes (A) the substrate; (B) the adhesive layer containing the PSA in contact with the substrate; (C) an optional first ink layer in contact with the substrate; and (D) an optional second ink layer in contact with the optional first ink layer. The optional first ink layer is a continuous layer and the optional second ink layer is a graphic image. In a further embodiment, the skin marker has the following Structure VI:

Optional Second Ink Layer/Optional First Ink Layer/Substrate/Adhesive Layer (Structure VI).

In Structures I-VI, the adjacent layers directly contact each other.

The adhesive layer may be formed on a release liner using an 8 path scraper tool, and then dried to remove all, or substantially all of the solvent in the adhesive composition. Then, the substrate, such as a nonwoven fabric, may be laminated to the adhesive layer using a hand laminator or a mechanical laminator. Optional physical or chemical treatments of the substrate are performed before lamination to the adhesive layer. Optional additives to the adhesive layer may be added by mixing or agitation, prior to application onto the release liner.

In some embodiments, the adhesive layer is applied to the substrate via slot die coating. The coated layer is then exposed to heat and convective air movement to remove, or substantially remove, solvent and/or water that may be present in the PSA. The slot die coating and heat/air movement exposure steps may be repeated until the desired thickness of the adhesive layer is attained.

In some embodiments, the adhesive layer is hot-melt coated on the substrate. During hot-melt coating, the PSA is placed in a drum unloader, in which the PSA is heated to a target temperature. The PSA is then pumped to a melt tank with a controlled temperature and volume level. A coating head pumps the PSA from the melt tank at a constant rate to maintain a constant layer thickness. The coated layer is then cooled to room temperature.

In an embodiment, a sheet is formed as described above. Then, the sheet may be die cut, hand cut, or sliced into the desired shape to form the skin marker.

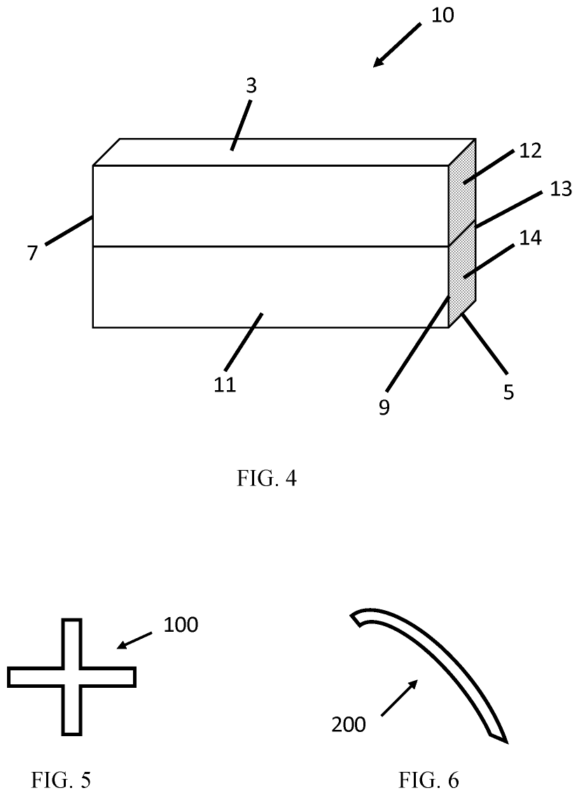

In an embodiment, as shown in FIG. 4, the skin marker 10 has a top facial surface 3 and an opposing bottom facial surface 5. The skin marker 10 has a front edge surface 7 and an opposing rear edge surface 9. The skin marker has a left edge surface 11 and an opposing right edge surface 13. The edge surfaces—including the front edge surface 7, rear edge surface 9, left edge surface 11, and right edge surface 13—may collectively be referred to herein as “sides,” “side edges,” or “edge surfaces” of the skin marker. The skin marker 10 shown in FIG. 4 has four sides, or four edge surface. It is understood that references to a “center” of a skin marker refers to a point that is equidistant between any two opposing sides (or edge surfaces) of the skin marker.

The skin marker has a shape. Nonlimiting examples of suitable shapes include polygon, circle, ovoid, X-shaped, cross-hair shaped, and irregular. A nonlimiting example of a polygon is a rectangle. FIG. 4 depicts a rectangular skin marker. In other embodiments, the skin marker is in the shape of an item, such as a flower, an animal, an emoticon, a letter, a number, a symbol, an imaginary creature (e.g., a unicorn), or the like.

In an embodiment, the skin marker has an X-shape. FIG. 5 depicts an X-shaped skin marker 100.

In an embodiment, the skin marker has an irregular shape. FIG. 6 depicts an irregular-shaped skin marker 200.

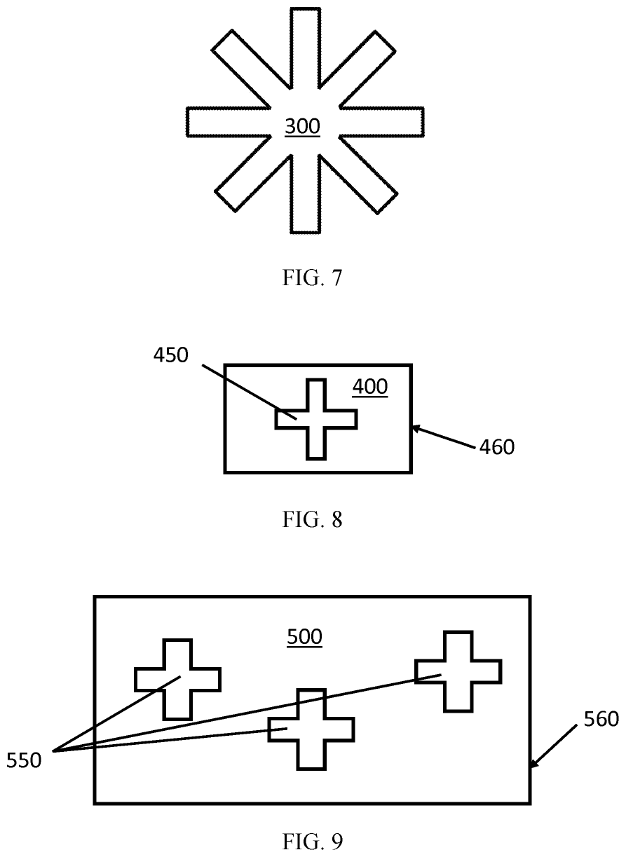

In an embodiment, the skin marker has a symbol shape, such as an asterisk. FIG. 7 depicts a symbol-shaped skin marker 300, which is in the shape of an asterisk.

The skin marker may be continuous from each edge surface to the opposing edge surface. A skin marker that is continuous excludes skin markers with voids, cutouts (also referred to herein as a “cutaway”), perforations, or the like. In other words, in a continuous skin marker, the top facial surface 3 and the opposing bottom facial surface 5, and any surface therebetween, extends from each edge surface to the opposing edge surface without any breaks, gaps, or voids within the skin marker's outer perimeter. FIGS. 4-7, 12, 14, 15, and 16A depict continuous skin markers. FIGS. 8, 9, 10, 13, 16B, and 17 depict skin markers that have cutouts and are therefore not continuous skin markers. In skin markers that have a cutaway, the cutaway area is not considered with respect to opacity, WVTR, or breathability measurements.

In an embodiment, the skin marker has a cutout (or a cutaway). The total area of the cutaway is less than the surface area of the skin marker. In embodiment, the area of the cutaway is less than 1 in², or less than 0.5 in², or less than 0.1 in², or less than 0.05 in². In a further embodiment, the area of the cutaway is from 0.01 in² to 1 in², or from 0.01 in² to 0.5 in², or from 0.01 in² to 0.1 in², or from 0.01 in² to 0.05 in², or from 0.01 in² to 0.04 in². In a further embodiment, the area of the cutaway is from 0.01 in², or 0.02 in², or 0.03 in² to 0.04 in², or 0.05 in², or 0.10 in², or 0.50 in², or 0.75 in², or 1.0 in².

In another embodiment, the skin marker 400 includes a cutaway 450 within the skin marker's outer perimeter 460, as shown in FIG. 8 A cutaway is a void extending from the top facial surface to the bottom facial surface of the skin marker. In other words, when the skin marker is positioned on a person's skin, the person's skin is visible and exposed to ambient environment at the cutaway.

The cutaway 450 may have a shape, which may be any shape disclosed herein. The skin marker 400 depicted in FIG. 8 includes an X-shaped cutaway 450 positioned within the skin marker's outer perimeter 460.

The skin marker may include more than one cutaway. FIG. 9 depicts a skin marker 500 having a rectangular shape, with three cutaways 550 positioned within the skin marker's outer perimeter 560.

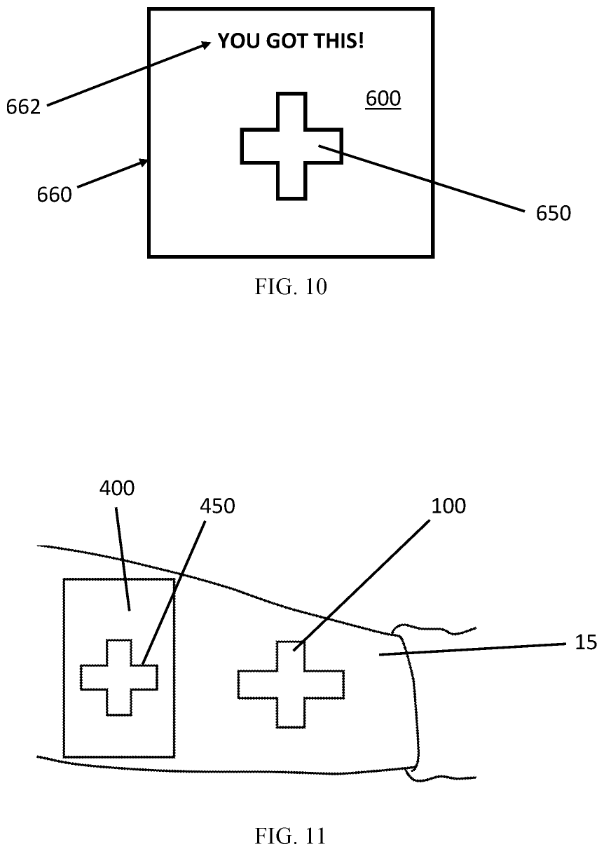

FIG. 10 depicts a skin maker 600 having a rectangular shape and a cutaway 650 positioned within the skin marker's outer perimeter 660. The skin marker 600 includes a graphic image 662 printed thereon. In FIG. 10, the graphic image 662 is in the form of text and symbols depicting a motivational message (YOU GOT THIS!).

In some embodiments, the skin marker includes a cutaway having a circular shape. In further embodiments, the circular cutaway is positioned in the center of the skin marker. A cutaway that is positioned in the center of the skin marker is positioned equidistant from each opposing surface edge of the skin marker.

The present skin marker is suitable for being applied to an individual's skin, including both human and animal skin. The present skin marker is suitable for being applied over a temporary tattoo, or another ink marking on an individual's skin, to protect said temporary tattoo or ink marking from being exposed to ambient environment and other conditions that would otherwise cause the temporary tattoo or ink marking to wear off of the individual's skin. The skin marker having an opacity of at least 65% prevents the individual wearing the skin marker, as well as other individuals, from seeing the temporary tattoo or the ink marking through the skin marker. Further, the skin marker with at least one of (i) a WVTR of at least 100 g/m²/day, or at least 400 g/m²/day, or at least 500 g/m²/day, or at least 750 g/m²/day, or at least 850 g/m²/day or (ii) a breathability of at least 1.0 grams mass lost after 24 hours at 50 °C, or at least 2.0 g mass lost after 24 hours at 50 °C makes the skin marker more comfortable for the individual to wear on their skin for extended period of time, such as over the course of a 6-week radiation therapy treatment.

In some embodiments, the skin marker includes a graphic image that is visible to a medical professional while the skin marker is being worn by an individual. The graphic image identifies the radiation treatment area for the patient. In such a case, the individual does not require a tattoo (permanent or temporary), nor do they require any other type of ink marking directly on their skin. Instead, the skin marker serves to identify the radiation treatment area. In this embodiment, the skin marker with an opacity of at least 65% prevents the medical professional for mistaking a mole or other marking on the individual's skin with the radiation treatment area identifier. Further, the skin marker with at least one of (i) a WVTR of at least 100 g/m²/day, or at least 400 g/m²/day, or at least 500 g/m²/day, or at least 750 g/m²/day, or at least 850 g/m²/day or (ii) a breathability of at least 1.0 grams mass lost after 24 hours at 50 °C, or at least 2.0 g mass lost after 24 hours at 50 °C makes the skin marker more comfortable for the individual to wear on their skin for extended period of time, such as over the course of a 6-week radiation therapy treatment. In some embodiments, where the skin marker is used as the radiation treatment area identifier, a medical professional may outline the marker with ink on the individual's skin, or place a temporary tattoo to outline the marker, for easy replacement in the event that the skin marker is accidentally removed before the end of the radiation therapy treatment.

FIG. 11 depicts an X-shaped skin maker 100 and a rectangular-shaped skin marker 400 with an X-shaped cutout 450 positioned on a person's skin surface 15. In FIG. 11, the skin surface 15 shown is a person's arm skin, but it is understood that the present skin maker may be positioned at any other skin surface on a person's body, such as on a leg, back, neck, or abdomen.

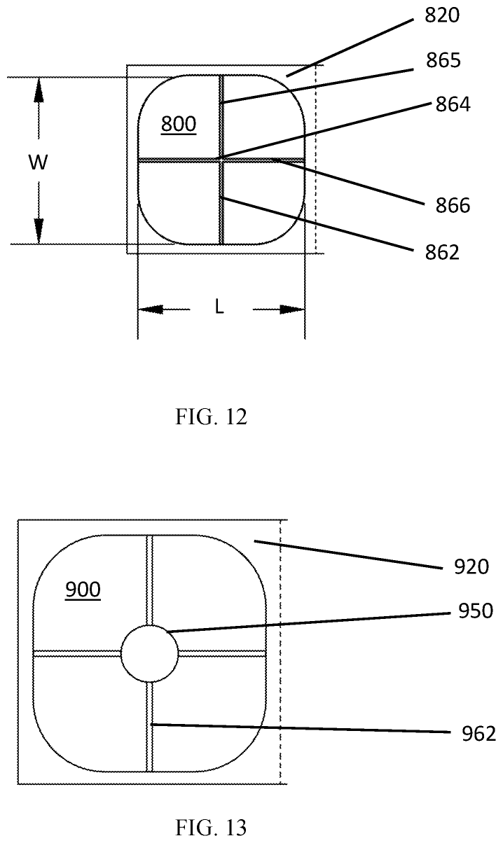

FIG. 12 depicts a squircle-shaped skin marker 800. A “squircle” is a square with four rounded corners, as shown in FIG. 12. The skin marker 800 includes a graphic image 862 printed thereon. In FIG. 12, the graphic image 862 is in the form of a crosshairs symbol, which includes two intersecting perpendicular lines 865 and 866, which intersect at the center point 864 of the skin marker 800. The skin marker 800 has a length, L, and a width, W. The skin maker 800 is positioned on a release liner 820.

The length, L, and width, W, of the skin marker may be equal or different. In an embodiment, the length, L, is equal to the width, W. In another embodiment, the length, L, is different than the width, W. In an embodiment, the length, L, is from 0.25 to 15 inches, or from 0.50 to 10 inches, or from 0.50 to 5 inches, or from 0.50 to 2 inches, or from 0.25 to 1 inch, or from 0.50 to 2 inches, or from 0.50 to 1 inch, or from 0.25 to 1.5 inches, or from 0.5 to 1.5 inches. In an embodiment, the width, W, is from 0.25 to 15 inches, or from 0.50 to 10 inches, or from 0.50 to 5 inches, or from 0.50 to 2 inches, or from 0.25 to 1 inch, or from 0.50 to 2 inches, or from 0.50 to 1 inch, or from 0.25 to 1.5 inches, or from 0.5 to 1.5 inches. In another embodiment, the length, L, is equal to the width, W, and each is from 0.25 to 15 inches, or from 0.50 to 10 inches, or from 0.50 to 5 inches, or from 0.50 to 2 inches, or from 0.25 to 1 inch, or from 0.50 to 2 inches, or from 0.50 to 1 inch, or from 0.25 to 1.5 inches, or from 0.5 to 1.5 inches.

FIG. 13 depicts a squircle-shaped skin marker 900 with a circle-shaped cutout 950. The skin marker 900 includes a graphic image 962 printed thereon. In FIG. 13, the graphic image 962 is in the form of a crosshairs symbol. The cutaway 950 is positioned in the center of the skin marker 900. The skin maker 900 is positioned on a release liner 920.

In an embodiment, the adhesive layer may be applied directly to an individual's skin, such as by spraying an aerosol adhesive onto an individual's skin, and then contacting the bottom surface of the substrate with the adhesive layer. Alternatively, the skin marker containing the substrate and the adhesive layer may be contacted with an individual's skin, such that a bottom surface of the adhesive layer directly contacts the individual's skin.

While the present disclosure is directed to radiation therapy skin markers, it is understood that the present skin marker may alternatively be used to identify treatment areas on a person for liposuction, filler injections, or other instances where a surgical marker would be used.

The skin marker may comprise two or more embodiments disclosed herein.

### E. Sheet

The present disclosure provides a sheet. The sheet contains (A) a plurality of skin markers and (B) a release liner in contact with the adhesive layer of each of the plurality of the skin markers.

The skin markers may be any skin marker disclosed herein. The release liner may be any release liner disclosed herein.

In an embodiment, each of the plurality of the skin markers has a plurality of side edges, and at least two side edges of each skin marker directly contacts a side edge of at least two skin markers.

In another embodiment, none of the side edges of the skin markers directly contacts a side edge of another skin marker.

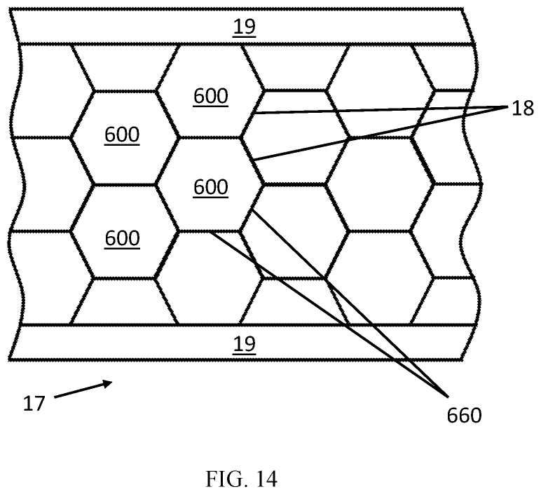

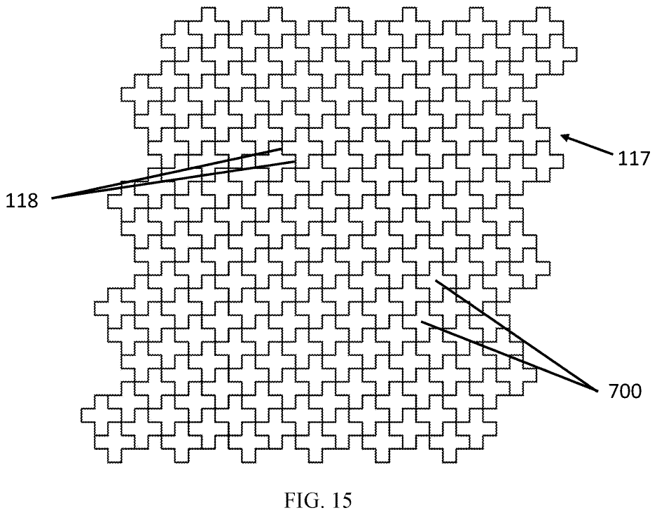

FIG. 14 depicts a sheet 17 with a plurality of skin markers 600. In FIG. 14, the skin markers 600 each have a hexagon shape, with each skin marker 600 having six side edges 18. The side edges 18 define the outer perimeter 660 of the skin marker 600. In the sheet 17, at least two side edges 18 of each skin marker 600 directly contacts a side edge 18 of at least two other skin markers 600.

The sheet may or may not include a side panel 19, which extends from one end of the sheet to the opposing end. The side panel 19 may facilitate the sheet 17 being fed through a printer. The sheet 17 of FIG. 14 has two side panels 19.

FIG. 15 depicts a sheet 117 with a plurality of skin markers 700, each skin marker 700 having an X-shape, with each skin marker 700 having twelve side edges 118. At least two side edges 118 of each skin marker 700 directly contacts a side edge 118 of at least two other skin markers 700.

The sheets 17 and 117 of FIGS. 14 and 15 depict skin markers 600, 700 that are arranged in a nested arrangement that minimizes waste of material.

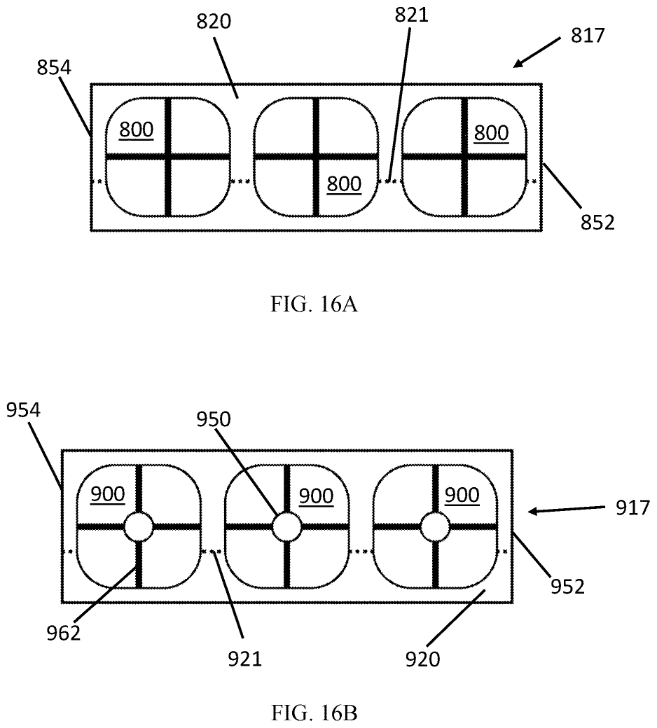

FIG. 16A depicts a sheet 817 with a plurality of skin markers 800, each skin marker 800 having a squircle-shape. Each skin marker 800 is positioned such that the adhesive layer contacts a release liner 820, but the skin markers 800 do not contact each other. The release liner 820 has a perforation line 821 extending across a length of the release liner, from a first end 852 to a second end 854 of the release liner 820.

FIG. 16B depicts a sheet 917 with a plurality of skin markers 900, each skin marker 900 having a squircle-shape and a circular shape cutaway 950 positioned within the skin marker's outer perimeter. Each skin marker 900 is positioned such that the adhesive layer contacts a release liner 920, but the skin markers 900 do not contact each other. The release liner 920 has a perforation line 921 extending across a length of the release liner, from a first end 952 to a second end 954 of the release liner 920. The skin marker 900 includes a graphic image 962 printed thereon in the form of a crosshairs symbol.

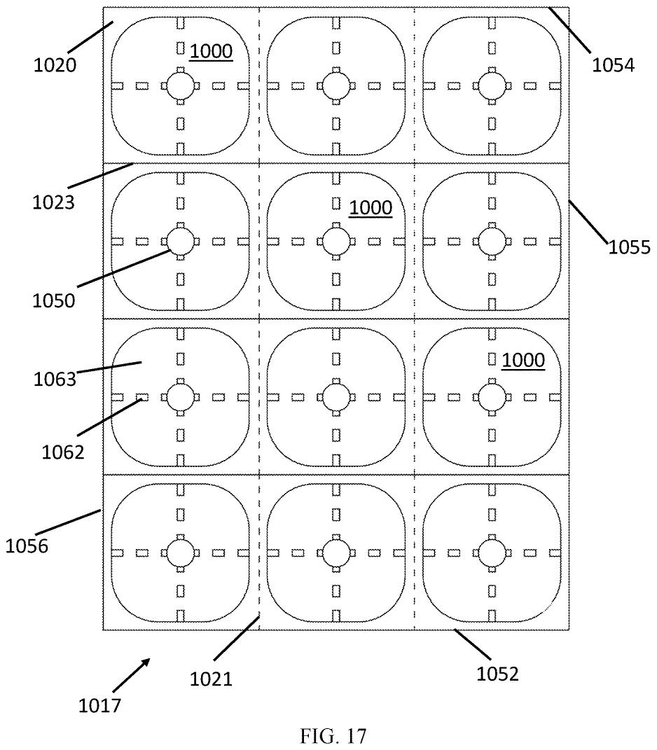

FIG. 17 depicts a sheet 1017 with a plurality of skin markers 1000, each skin marker 1000 having a squircle-shape and a circular shape cutaway 1050 positioned within the skin marker's outer perimeter. Each skin marker 1000 is positioned such that the adhesive layer contacts a release liner 1020, but the skin markers 1000 do not contact each other. The release liner 1020 has (i) a first perforation line 1021 extending across a length of the release liner 1020, from a first end 1052 to an opposing second end 1054 of the release liner 1020 and (ii) a second perforation line 1023 extending across a width of the release liner 1020, from a third end 1055 to an opposing fourth end 1056 of the release liner 1020. The skin marker 900 includes a graphic image 1062 printed thereon in the form of a crosshairs symbol, printed in dashed (or broken) lines. The graphic image 1062 is a second ink layer, which has be printed on a first ink layer 1063 that is a continuous ink layer. It is understood that the first ink layer 1063 is not visible, or not substantially visible, where it is covered by the second ink layer 1062.

The sheet may comprise two or more embodiments disclosed herein.

By way of example, and not limitation, examples of the present disclosure are provided.

### Examples

### Materials

The materials used in the examples are provided in Table 1A and 1B. In Table 1B, “total thickness” refers to the cumulative thickness of the material, with the release liner removed.

TABLE 1A

| Name | Description | Source |
|---|---|---|
| IZ1004 | Skin marker for radiation therapy; release liner; | IZI Medical Products |
| | clear backing with two intersecting perpendicular | |
| | blue lines printed thereon; adhesive layer; | |
| | gradual octagon shape with a 1.4 cm diameter; | |
| | surface area = 1.9470 cm² | |
| PointGuards 315 | Skin marker for radiation therapy; clear circle; | Beekley Corporation |
| | adhesive layer; 2.5 cm diameter | |
| Nexcare Tegaderm | Transparent dressing; adhesive layer | 3M |
| ARcare 93690 | Medical grade pressure-sensitive acrylic adhesive | Adhesives Research, |
| | coated on a stretchable nonwoven fabric; release | Inc. |
| | liner thickness = 50 μm | |
| ARcare 93439 | Medical grade pressure-sensitive adhesive coated | Adhesives Research, |
| | on a white PET nonwoven fabric; release liner | Inc. |
| | thickness = 50 μm | |
| MED 5718P | Single-coated white, stretchable, PET polyester | Avery Dennison |
| | nonwoven with a porous acrylic adhesive; | Corporation |
| | release liner thickness = 76 μm | |
| MED 5744 | Single-coated, white PET polyester nonwoven | Avery Dennison |
| | with an acrylic-based adhesive; release liner | Corporation |
| | thickness = 76 μm | |
| dermaFLEX V 400 F | White flexible vinyl with H-566 acrylic | FLEXcon |
| White H-566 50K-6 | adhesive; release liner thickness = 3.1 mil; | |
| | Product #FLX062422 | |
| dermaFLEX NWP 240 | Nonwoven polyester fabric with H-778 adhesive; | FLEXcon |
| White H-778 90PFW | release liner thickness = 6.9 mil; Product | |
| | #FLX068292 | |
| 4077 SC | Elastic extended wear nonwoven tape; white | 3M |
| | elastic nonwoven substrate with tackified acrylic- | |
| | based adhesive; release liner thickness = 0.15 mm | |
| 4576 | Polyester spunlace nonwoven tape with 3M | 3M |
| | medical extended wear acrylic adhesive; release | |
| | liner thickness = 0.09 mm | |
| 4578 | Polyester spunlace nonwoven extended wear | 3M |
| | tape; 100% polyester white spunlace nonwoven | |
| | substrate with 3M extended wear acrylate | |
| | adhesive; release liner thickness = 0.08 mm | |
| 2476P | Single-sided polyester spunlace nonwoven tape; | 3M |
| | white polyester spunlace nonwoven with medical | |
| | silicone adhesive; release liner thickness = 0.08 mm | |
| 4076 SC | Spunlace extended wear nonwoven tape; white | 3M |
| | spunlace polyester nonwoven with tackified | |
| | acrylic-based adhesive; release liner thickness = | |
| | 0.15 mm | |
| Polyken 2328F | Non-woven single-coated tape; white spunlace | Berry Global Inc. |
| | PET nonwoven with acrylic adhesive; release | |
| | liner thickness = 0.173 mm | |
| Polyken 3598B | Single-coated film tape; PET polyester film with | Berry Global Inc. |
| | medical-grade acrylic adhesive; release liner | |
| | thickness = 0.150 mm | |
| Mactac TM8101 | White spunlace nonwoven fabric with a medial | Morgan Adhesives |
| | grade acrylic adhesive; release liner thickness = | Company LLC |
| | 101.6 μm | |
| AROSET PS 5333 | Pressure sensitive adhesive; medical-grade | Ashland Inc. |
| | adhesive; self-crosslinking solution acrylic; | |
| | solids = 57 wt %; also referred to as AROSET | |
| | PS 6464 | |
| 4075 | Transfer pressure sensitive adhesive; 3M medical | 3M |
| | tackified acrylic adhesive thickness = 0.07 mm; | |
| | release liner thickness = 0.05 mm | |
| Release Liner | Fluoropolymer coated polyester release liner; | 3M |
| | transparent polyester; product number 9956 | |
| EVALON AP Paper | 58 grams per square meter (gsm); non-woven | Freudenberg |
| Sheet (58 gsm) | polyester/nylon microfiber paper; non-stretch; | Performance Materials |
| | microfilament spunbond non-woven fabric; | |
| | white; sheet size = 22 inch × 30 inch; | |
| | thickness = 0.176 mm | |
| EVALON AP Paper | 100 gsm; non-woven polyester/nylon microfiber | Freudenberg |
| Sheet (100 gsm) | paper; non-stretch; microfilament spunbond non- | Performance Materials |
| | woven fabric; white | |
| Fitesa (25 gsm) | 25 gsm; non-woven poly lactic acid (PLA); | Fitesa S.A. |
| | spunbond non-woven fabric; white | |
| Fitesa (30 gsm) | 30 gsm; non-woven poly lactic acid (PLA); | Fitesa S.A. |
| | spunbond non-woven fabric; white | |
| Siegwerk PRO BLACK | water-based black ink | Siegwerk Druckfarben |
| | | AG & Co. KGaA |
| SunCure Accuflex | flesh tone UV-curable flexographic ink system | Sun Chemical |

TABLE 1B

| | | Substrate | | | Adhesive | | Total | |
|---|---|---|---|---|---|---|---|---|
| Material | Substrate | Thickness | | Adhesive | Thickness | | Thickness | |
| ARcare 93690 | Nonwoven | 178 | μm | Acrylic | 69 | μm | 247 | μm |
| | fabric | | | adhesive | | | | |
| ARcare 93439 | Nonwoven | 279 | μm | Medical- | 66 | μm | 345 | μm |
| | PET fabric | | | grade | | | | |
| | | | | adhesive | | | | |
| MED 5718P | PET | 190 | μm | Acrylic | 50 | μm | 240 | μm |
| | nonwoven | | | adhesive | | | | |
| MED 5744 | PET | 330 | μm | Acrylic | 102 | μm | 432 | μm |
| | nonwoven | | | adhesive | | | | |
| dermaFLEX V | vinyl | 3.5 mil | | H-566 acrylic | 1.9-2.1 mil | | 5.4-5.6 mil | |
| 400 F | | (88.9 μm) | | adhesive | (48.3-53.3 μm) | | (137-142 μm) | |
| dermaFLEX | Nonwoven | 18.0 mil | | H-778 | 1.9-2.1 mil | | 19.9-20.1 mil | |
| NWP 240 | polyester | (457 μm) | | adhesive | (48.3-53.3 μm) | | (505-510 μm) | |
| | fabric | | | | | | | |
| 3M 4077 SC | Elastic | 0.16 | mm | Tackified | 0.07 | mm | 0.23 | mm |
| | nonwoven | | | acrylic | | | | |
| | | | | adhesive | | | | |
| 3M 4576 | Polyester | 0.30 | mm | 3M acrylic | 0.10 | mm | 0.40 | mm |
| | spunlace | | | adhesive | | | | |
| | nonwoven | | | | | | | |
| 3M 4578 | Polyester | 0.25 | mm | 3M acrylate | 0.10 | mm | 0.35 | mm |
| | spunlace | | | adhesive | | | | |
| | nonwoven | | | | | | | |
| 3M 2476P | Polyester | 0.25 | mm | Silicone | 0.10 | mm | 0.35 | mm |
| | spunlace | | | adhesive | | | | |
| | nonwoven | | | | | | | |
| 3M 4076 SC | Polyester | 0.25 | mm | Tackified | 0.071 | mm | 0.325 | mm |
| | spunlace | | | acrylic | | | | |
| | nonwoven | | | adhesive | | | | |
| Polyken 2328F | Spunlace | 0.356 | mm | Acrylic | 0.046 | mm | 0.574 | mm |
| | PET | | | adhesive | | | | |
| | nonwoven | | | | | | | |
| Polyken 3598B | PET film | 0.025 | mm | Acrylic | 0.036 | mm | 0.211 | mm |
| | | | | adhesive | | | | |
| Mactac | Spunlace | 0.19 | mm | Acrylic | 0.046 | mm | 240.8 | μm |

| | | |
|---|---|---|
| TM8101 | nonwoven | adhesive |
| | fabric | |

### Preparation of Example A

Using a 3 mil (76.2 μm) 8 path scraper tool, a layer of pressure sensitive adhesive (AROSET PS 5333) is applied to the release surface of the release liner. The adhesive-coated release liner is then placed in an oven for five minutes at 300 °F; after which it is cooled to room temperature. Then, adhesive-coated release liner is laminated to a piece of EVALON AP Paper Sheet (100 gsm) substrate using a hand laminator (4.5 pound rubber roller) to form Example A. The adhesive layer of Example A has a thickness of 1 mil (25.4 μm) (16 pounds per ream (lbs/rm)).

### Preparation of Comparative Sample 1 (CS 1)

The release liner is cut into 6 inch×8 inch pieces. A 6 inch×8 inch piece of EVALON AP Paper Sheet (58 gsm) is cut.

Using an 8 Path Wet Film Applicator from Precision Gage & Tool Co., a 6 mil (152.4 μm) layer of pressure sensitive adhesive (AROSET PS 5333) is applied to a surface of the cut release liner. The adhesive-coated release liner is then placed in an oven for five minutes at 300 °F; after which it is cooled to room temperature. Then, the cut EVALON AP Paper Sheet (58 gsm) substrates are placed in contact with the adhesive layer and a weighted 4.5 pound rubber roller is passed over the substrate to form a sheet of skin markers.

The adhesive layer of Comparative Sample 1 has a thickness of 6 mil.

### Preparation of Example B

A 6 inch×8 inch piece of EVALON AP Paper Sheet (100 gsm) substrate is cut and laminated to a 6 inch×8 inch piece of 3M 4075 adhesive/release liner using a hand laminator (4.5 pound rubber roller) to form Example B.

### Preparation of Example C

EVALON AP Paper Sheet (100 gsm) substrate laminated to a piece of 3M 4075 adhesive/release liner using a machine laminator to form Example C. The sample is then printed first with a skin tone ink (SunCure Accuflex), and is then printed with cross hairs (black Siegwerk PRO BLACK).

The structures of Examples A-C and CS 1 are summarized in Table 1C.

TABLE 1C

| Example | Substrate | Adhesive | Adhesive Thickness | |
|---|---|---|---|---|
| Example A | Evalon | AROSET PS | 1 mil | (25.4 μm) |
| | 100 gsm | 5333 | | |
| CS 1 | Evalon | AROSET PS | 6 mil | (152.4 μm) |
| | 58 gsm | 5333 | | |
| Example B | Evalon | 3M 4075 | 2.76 mil | (70.1 μm) |
| | 100 gsm | | | |
| Example C | Evalon | 3M 4075 | 2.76 mil | (70.1 μm) |

100 gsm

### Thickness

Thickness is measured using a Mitituoyo micrometer. For samples of spunbound non-woven fabric (i.e., Fitesa or EVALON AP Paper), samples are cut from the fabric including areas visually identified as thick, medium, and thin areas. The results are reported in Table 2 in mils.

TABLE 2
Thickness (mils)

| | | | | | | | | Standard | Standard |
|---|---|---|---|---|---|---|---|---|---|
| | | Number | | | Std. | | | Deviation | Deviation |
| | | of | | Std. | Error | Lower | Upper | Lower | Upper |
| Substrate | Area | Samples | Mean | Dev. | Mean | 95% | 95% | 95% | 95% |
| Evalon | Thin | 10 | 5.34 | 0.26 | 0.08 | 5.15 | 5.52 | 0.18 | 0.47 |
| 58 gsm | Medium | 10 | 6.94 | 0.36 | 0.11 | 6.68 | 7.20 | 0.25 | 0.66 |
| | Thick | 10 | 10.64 | 0.65 | 0.21 | 10.17 | 11.10 | 0.45 | 1.19 |
| Evalon | Thin | 10 | 8.87 | 0.50 | 0.16 | 8.51 | 9.23 | 0.34 | 0.91 |
| 100 gsm | Medium | 10 | 10.83 | 0.40 | 0.13 | 10.54 | 11.11 | 0.27 | 0.72 |
| | Thick | 10 | 13.92 | 1.21 | 0.38 | 13.05 | 14.78 | 0.83 | 2.20 |
| Fitesa | Thin | 5 | 3.26 | 0.33 | 0.15 | 2.85 | 3.67 | 0.20 | 0.95 |
| 25 gsm | Thick | 5 | 5.45 | 0.86 | 0.39 | 4.38 | 6.52 | 0.52 | 2.48 |
| Fitesa | Thin | 5 | 4.34 | 0.49 | 0.22 | 3.74 | 4.94 | 0.29 | 1.39 |
| 30 gsm | Thick | 5 | 6.4 | 0.25 | 0.11 | 6.08 | 6.72 | 0.25 | 0.73 |

### Opacity

Opacity is measured using a Leneta opacity chart (Form N2C-2 Opacity, having a white half and a black half) and an X-Rite Pantone eXact spectrophotometer (Contrast Ratio Method, Illuminant: D65, Observer: 2°, Filters: M0(no)), using a spot measurement area size of 4 mm. Using the spectrophotometer, the absorbance (Y) is measured against the sample against the black area of the chart and against the white area of the chart. The opacity (or contrast ratio) is calculated using the following Equation A:

$$Opacity = ( \frac{{Y}_{white}}{{Y}_{black}} ) \times 100. \qquad Equation A$$

Samples are mounted to the charts at the line between the black and white portions of the chart, with half of the sample adhered to the black portion of the chart and the other half adhered to the white portion of the chart.

For IZ1004, the adhesive surface of the sample is adhered to the chart after being removed from its release liner. Opacity is measured in sections of the IZ1004 that do not include the two intersecting perpendicular blue lines.

For samples of spunbound non-woven fabric (i.e., Fitesa or EVALON AP Paper), samples are cut from the fabric from areas visually identified as thick, medium, and/or thin areas, as provided in Table 3A. These samples without adhesive are not adhered to the Leneta chart, but simply placed on top of the Leneta chart while measuring opacity.

For Examples A-C and CS 1, prepared as described above, five samples are cut. The release liner is removed and the sample is adhered to the chart, with the adhesive layer contacting the chart.

Results are reported in Tables 3A and 3B.

TABLE 3A
Opacity (%)

| | | | | | | | | Standard | Standard |
|---|---|---|---|---|---|---|---|---|---|
| | | Number | | | Std. | | | Deviation | Deviation |
| | | of | | Std. | Error | Lower | Upper | Lower | Upper |
| Substrate | Area | Samples | Mean | Dev. | Mean | 95% | 95% | 95% | 95% |
| Evalon | Thin | 10 | 67.92 | 5.81 | 1.84 | 63.76 | 72.08 | 4.00 | 10.61 |
| 58 gsm | Medium | 10 | 84.20 | 2.41 | 0.76 | 82.47 | 85.93 | 1.66 | 4.40 |
| | Thick | 10 | 94.95 | 0.90 | 0.28 | 94.31 | 95.59 | 0.62 | 1.64 |
| Evalon | Thin | 10 | 89.97 | 2.18 | 0.69 | 88.41 | 91.53 | 1.50 | 3.98 |
| 100 gsm | Medium | 10 | 93.11 | 1.05 | 0.33 | 92.36 | 93.86 | 0.72 | 1.92 |
| | Thick | 10 | 96.46 | 0.59 | 0.19 | 96.04 | 96.88 | 0.40 | 1.07 |
| Fitesa | Thin | 5 | 11.94 | 1.23 | 0.55 | 10.41 | 13.47 | 0.74 | 3.53 |
| 25 gsm | Thick | 5 | 28.5 | 2.23 | 1.00 | 25.73 | 31.27 | 1.34 | 6.41 |
| Fitesa | Thin | 5 | 25.86 | 1.67 | 0.75 | 23.78 | 27.94 | 1.00 | 4.81 |
| 30 gsm | Thick | 5 | 38.2 | 2.86 | 1.28 | 34.65 | 41.75 | 1.71 | 8.21 |

TABLE 3B
Opacity (%)

| Example | Number of Samples | Mean | Standard Deviation |
|---|---|---|---|
| Example A | 5 | 97.28 | 0.44 |
| CS 1 | 5 | 54.93 | 6.90 |
| Example B | 5 | 67.84 | 6.64 |
| Example C | 5 | 89.07 | 2.51 |
| IZ1004 | 10 | 4.33 | 0.24 |
| PointGuards 315 | 5 | 6.22 | 0.06 |
| Nexcare Tegaderm | 5 | 8.02 | 1.30 |
| dermaFLEX V 400 F | 10 | 95.17 | 0.88 |
| dermaFLEX NWP 240 | 10 | 64.89 | 2.96 |
| 3M 4077 SC | 10 | 57.73 | 2.59 |
| 3M 4576 | 10 | 46.87 | 6.81 |
| 3M 4578 | 10 | 43.15 | 3.92 |
| 3M 2476P | 10 | 43.39 | 3.32 |
| 3M 4076 SC | 10 | 45.99 | 2.48 |
| Polyken 2328F | 10 | 61.77 | 3.99 |
| Polyken 3598B | 10 | 1.23 | 0.07 |
| Mactac TM8101 | 10 | 41.46 | 2.60 |
| ARcare 93690 | 10 | 46.02 | 2.57 |
| Arcare 93439 | 10 | 43.65 | 4.94 |
| MED 5718P | 10 | 44.13 | 3.36 |
| MED 5744 | 10 | 42.49 | 5.24 |

### Breathability and WVTR

Samples are die-cut into circles with a mallet die sample cutter available from Thwing-Albert to have a diameter of 2.9375 inches (7.46125 cm). The testable area of the samples in the Vapometer cups is 3,167 square millimeters (63.5 mm diameter test area).

The release liner is removed and the examples are placed over the opening of a Thwing-Albert Model 68-3000 EZ-Cup Vapometer filled with 100 grams of deionized water, adhesive-side down. After being weighed using a Sartorius balance, the Vapometer and example are placed in a 50 °C oven for 24 hours. After being removed from the oven, the Vapometer and the example are allowed to cool to room temperature and then the weight of the Vapometer and example are taken again. The total grams of water lost is calculated by subtracting the weight of the covered Vapometer after the oven (50 °C/24 hours) from the weight of the covered Vapometer before the oven. The average of three samples is reported. Breathability results are reported in Table 4A.

In Table 4A, WVTR is extrapolated based on the measured mass lost after 24 hours at 50 °C, or is reported based on supplier-supplied WVTR values.

TABLE 4A
Breathability (63.5 mm test area)

| | Mass Lost (g) after | WVTR |
|---|---|---|
| Example or Material | 24 hours @ 50 °C | (g/m²/day) |
| Example A | 8.167 | 977.8 |
| dermaFLEX V 400 F | 0.369 | |
| dermaFLEX NWP 240 | 1.433 | |
| 3M 4077 SC | 1.222 | |
| 3M 4576 | 0.945 | |
| 3M 4578 | 1.172 | |
| Polyken 2328F | 11.518 | |
| Mactac TM8101 | 7.161 | |
| ARcare 93690 | 5.272 | |
| MED 5718P | 16.968 | |
| MED 5744 | 0.565 | |
| IZ1004 | 0.879 | |
| Evalon 58 gsm | 45.352 | |
| Evalon 100 gsm | 43.81 | |

WVTR in Table 4B is measured in accordance with ASTM E96 using the Desiccant Cup Method at 100% relative humidity and 37.8 °C. All samples are analyzed with a test area of 1 cm², except that the Nexcare Tegaderm was measured with a test area of 5 cm². The results reported are the average of three samples, except for the Nexcare Tegaderm (average of two samples).

TABLE 4B
WVTR (ASTM E96)

| Example or Material | WVTR (g/m²/day) |
|---|---|
| IZ1004 | 230 |
| PointGuards 315 | 465 |
| Nexcare Tegaderm | 571 |
| CS 1 | 1,846 |
| Example B | 450 |
| Example C | 858 |

As shown in Table 3B3, Examples A-C each exhibit the combination of an opacity of at least 6500 and a WVTR of at least 100 g/ma/day, suggesting that they are suitable skin markers. In contrast, while CS 1 exhibits a suitable WVTR of 1,846 g/ma/day, it has an opacity of less than 650% (specifically, of 54.930%), making it unsuitable for skin marker applications. Likewise, none of the other comparative samples tested achieved the desired combination of (i) high opacity (i.e., at least 650%) and (ii) a high WVTR (i.e., at least 100 g/m²/day) and/or a high breathability (i.e., at least 1.0 g mass lost after 24 hours at 50 °C), making these comparative samples unsuitable for skin markers applications.

It is specifically intended that the present disclosure not be limited to the embodiments and illustrations contained herein, but include modified forms of those embodiments including portions of the embodiments and combinations of elements of different embodiments as come within the scope of the following claims.

We claim:

1. A skin marker comprising:
(A) a substrate; and
(B) an adhesive layer comprising a pressure sensitive adhesive in contact with the substrate;
the skin marker having (i) a water vapor transmission rate of at least 100 g/m²/day and (ii) an opacity of at least 65%.

2. The skin marker of claim 1, wherein the skin marker has a water vapor transmission rate of at least 450 g/m²/day.

3. The skin marker of claim 1, wherein the skin marker has an opacity of at least 65% across an entire surface area of the skin marker.

4. The skin marker of claim 1, wherein the skin marker has an opacity of at least 75%.

5. The skin marker of claim 1, wherein the substrate has a water vapor transmission rate of at least 2,000 g/m²/day.

6. The skin marker of claim 1, wherein the substrate has an opacity of at least 30%.

7. The skin marker of claim 1, wherein the skin marker has a total thickness of from 2 mil to 20 mil.

8. The skin marker of claim 1, wherein the substrate is a nonwoven.

9. The skin marker of claim 1, wherein the substrate is a nonwoven comprising fiber formed from the group consisting of polypropylene, polyester, nylon, polyethylene, polylactic acid, and combinations thereof.

10. The skin marker of claim 1, wherein the adhesive layer further comprises an additive selected from the group consisting of calcium carbonate, titanium dioxide, and combinations thereof.

11. The skin marker of claim 1, wherein the adhesive layer comprises up to 20 wt % additive, based on a total weight of the adhesive layer.

12. The skin marker of claim 1, wherein a top surface of the substrate is printable.

13. The skin marker of claim 1, further comprising
(C) a topcoat in contact with a top surface of the substrate.

14. The skin marker of claim 13, wherein the topcoat is printable.

15. A sheet comprising:
(A) a plurality of the skin markers of claim 1; and
(B) a release liner in contact with the adhesive layer of each of the plurality of the skin markers.

16. The sheet of claim 15, wherein each of the plurality of the skin markers has a plurality of side edges; and
at least two side edges of each skin marker directly contact a side edge of at least two skin markers.

17. A skin marker comprising:
(A) a substrate; and
(B) an adhesive layer comprising a pressure sensitive adhesive in contact with the substrate;
the skin marker having (i) a breathability of at least 1.0 grams mass lost after 24 hours at 50 °C and (ii) an opacity of at least 65%.

18. The skin marker of claim 17, wherein the skin marker has a breathability of at least 2.0 grams mass lost after 24 hours at 50 °C.

19. The skin marker of claim 17, wherein the skin marker has a total thickness of from 2 mil to 20 mil.

20. A sheet comprising:
(A) a plurality of the skin markers of claim 17; and
(B) a release liner in contact with the adhesive layer of each of the plurality of the skin markers.
